(19) United States
(12) Patent Application Publication
(10) Pub. No.: US 2023/0384324 A1
(43) Pub. Date: Nov. 30, 2023

(54) FOCAL ADHESION COMPLEX PROTEINS AS A DIAGNOSTIC MARKER FOR PODOCYTOPHATHIES

(71) Applicant: Northwestern University, Evanston, IL (US)

(72) Inventors: Lorenzo Gallon, Chicago, IL (US); Robert V. Gerbasi, Chicago, IL (US)

(21) Appl. No.: 18/250,780

(22) PCT Filed: Nov. 11, 2021
(86) PCT No.: PCT/US21/58910

§ 371 Date: Apr. 27, 2023

Related U.S. Application Data
(60) Provisional application No. 63/112,473, filed on Nov. 11, 2020.

(51) Int. Cl. G01N 33/68

(52) U.S. Cl. CPC G01N 33/6893; G01N 2333/70546; G01N 2800/347

(57) ABSTRACT

A system and method for identifying and/or predicting damage or disease of a patient's kidney podocyte layer, wherein the system includes a test kit configured to analyze the plasma or urine of the patient; and identify the presence of focal adhesion complex proteins, or anti-bodies associated with the focal adhesion complex proteins, in the plasma or urine, wherein the focal adhesion complex proteins comprise Integrin, Talin, Filamin, Vinculin, or other focal adhesion complex proteins. Wherein the presence of the focal adhesion complex proteins indicates damage or disease to the patient's podocyte layer and the presence of the associated anti-bodies may be used to predict the likelihood of developing damage.

### FIELD

This disclosure is directed to a system using focal adhesion complex proteins from bodily fluids to identify patients with kidney podocyte pathologies, including patients with focal segmental glomerulosclerosis (FSGS), minimal change disease (MCD), and other pathologies that trigger similar nephrotic syndromes, and methods of using the same.

### BACKGROUND

The primary function of the kidney is to filter the blood. Each kidney contains approximately 1 million specialized structures called glomeruli, which function as entry and exit points for pressurized blood that is filtered through a glomeruluar filtration barrier to make urine. The glomerular filtration barrier is composed of an endothelial layer, a glomerular basement layer, and a podocyte layer. The podocyte is a terminally differentiated, non-replicating cell type, that is necessary to maintain glomerular structure and function. During certain nephrotic disease states caused by FSGS, MCD, viral infection, drug reaction, and genetic predisposition, podocytes begin to retract, as part of a process called podocyte effacement. As a consequence of podocyte effacement, proteins are lost from the blood into the urine, and patients eventually develop edema, high cholesterol, high blood pressure, and kidney failure.

Currently, no diagnostic test exists to identify patients with podocyte damage and effacement. Instead, patients with nephrotic syndrome undergo invasive and expensive biopsies that must be read by a pathologist. Patients respond favorably to immunosuppression in some cases, but evaluating the effectiveness of treatment is difficult without a diagnostic or prognostic indicator. Furthermore, different renal diseases that affect podocytes (FSGS and MCD) present similarly with proteinuria and foot process effacement on podocytes but respond drastically different to currently used immunosuppressive strategies. It would be beneficial to have a diagnostic biomarker that would allow physicians to identify patients with proteinuric nephropathies secondary to podocyte damage and be able to distinguish the etiology of these disease without the need for invasive biopsy procedures. This biomarker could also be used to clinically guide the type of immunosuppressive therapy to be given to patients and also used as a prognostic indicator for response to therapy.

### SUMMARY

A system and method for identifying and/or predicting damage or disease of a patient's kidney podocyte layer, wherein the system includes a test kit configured to analyze the plasma or urine of the patient; and identify the presence of focal adhesion complex proteins, or anti-bodies associated with the focal adhesion complex proteins, in the plasma or urine, wherein the focal adhesion complex proteins comprise Integrin, Talin, Filamin, Vinculin, or other focal adhesion complex proteins. Wherein the presence of the focal adhesion complex proteins indicates damage or disease to the patient's podocyte layer and the presence of the associated anti-bodies may be used to predict the likelihood of developing damage.

### BRIEF DESCRIPTION OF THE DRAWINGS

FIG. 1A-B: FIG. 1(A) is a proteomic analysis of plasma from 16 controls (left side) and 3 patients with active FSGS (far right). Red indicates the presence of proteins and black indicates the absence of detection. Hierarchical clustering identified the focal adhesion complex proteins (Integrin, Talin, Filamin, and Vinculin) in the FSGS plasma but not in controls. FIG. 1B is an immunoblot from control and FSGS plasma samples for FAC proteins including integrins, talin, and vinculin. The common plasma protein transferring was probed as a loading control.

FIG. 2 is an immunoblot of the focal adhesion complex protein Talin from Hela cell extract, control plasma (patients that have received a transplant but do not have FSGS), or primary FSGS patients. The blot shows that Talin is restricted to the FSGS patient samples and absent from the control patient samples.

FIG. 3 is a representation of plasma proteins that were subjected to Top-12 immunodepletion (a method designed to reduce the top-12 most abundant proteins from plasma or sera). Plasma proteins were either loaded directly onto an SDS-PAGE gel (1 uL Plasma (without immunodepletion)), 20 uL of immunodepleted plasma, or 10 uL of immunodepleted plasma. At 10 uL of immunodepleted plasma, the most abundant plasma proteins were reduced in concentration. Proteomic analysis was performed from 10 uL of immunodepleted plasma or sera.

FIG. 4 is a heatmap of immunodepleted proteins identified across plasma and sera from various samples.

FIG. 5A-E: (A) shows plasma from a patient with FSGS (previously confirmed to contain focal adhesion complex by mass spectrometry and immunoblotting), which was subjected to purification over either streptavidin beads (negative control) or protein A/G beads (to purify antibodies from plasma). Consistent with the binding activity of protein A/G, the purification precipitated antibody heavy and light chains (visualized by denaturing SDS-polyacrylamide gel electrophoresis). As expected, antibody heavy and light chains were absent from the streptavidin beads purification. FIGS. 5(B) and (C) show HELA cell lysates and the purifications, that were described in 5(A), which were subjected to immunoblotting and probed for either human Talin (B) or human Vinculin 5(C), or alpha-2B integrin 5(F). Protein A/G purifications were performed to co-purify Talin and Vinculin, whereas the streptavidin control purifications were absent focal adhesion components Talin and Vinculin. FIGS. 5(D) and (E) show Plasma from an FSGS patient subjected to protein A/G purification and analyzed by tandem mass spectrometry. Vinculin peptide spectra 5(D) and Actinin peptide spectra 5(E) were identified in the protein A/G purification but not the streptavidin purification.

Referring to FIG. 6, a working model of podocyte detachment during FSGS is presented. Top left-normal podocyte associated with GBM. Antibodies and other potential factors dissociate the foot processes from the basement membrane resulting in effacement (retraction and pruning of the podocyte. Podocyte proteins are absorbed back into the efferent arteriole and detectable in circulation.

### DETAILED DESCRIPTION

A test kit and method that may be used to identify a group of highly specific proteins, specifically focal adhesion complex proteins, including but not limited to Integrin, Talin, Filamin, Vinculin, present in the plasma of patients experiencing podocyte effacement is provided. It has been determined that, surprisingly, focal adhesion complex proteins are not present in control plasma populations or populations of patients with related nephrotic syndromes including membranous nephropathy, IgA nephropathy, minimal change disease, and genetic forms of FSGS (non-idiopathic forms). Therefore, the test kit and method would provide the first non-invasive laboratory test for podocytopathies, eliminating or reducing the need for diagnosis using an invasive kidney biopsy, and allowing for faster and, potentially, more sensitive and specific diagnosis for patients.

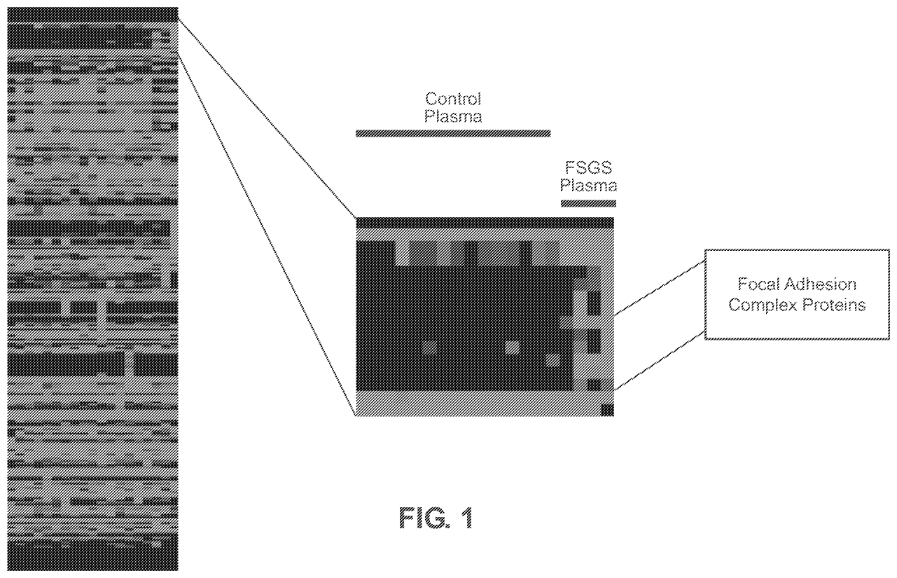

In one embodiment, a protein detection test kit and method is provided to identify focal adhesion complex proteins from the bodily fluids of patients with nephrotic syndrome. As shown in FIG. 1, hierarchical clustering identified the focal adhesion complex proteins (Integrin, Talin, Filamin, and Vinculin) in the sample including FSGS plasma (far right) but not in control samples (left side). This result indicates that focal adhesion complex proteins are a specific signature present in FSGS and can be used as a diagnostic or prognostic marker.

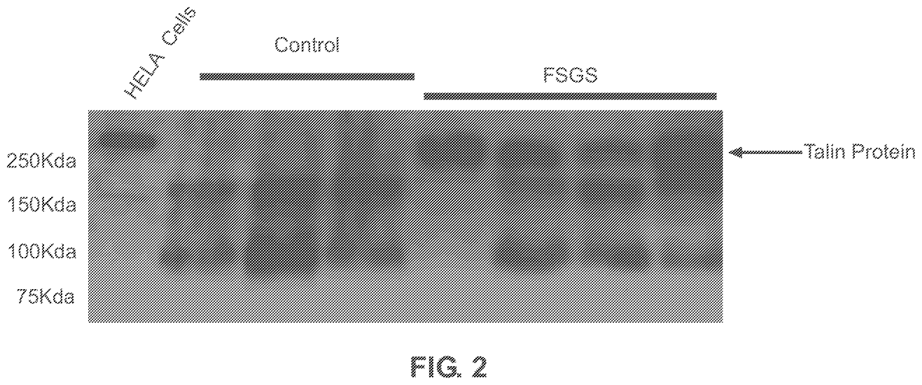

In one experiment, three different control plasma samples were loaded (Control) and four different FSGS patient plasma samples (FSGS) were loaded (far right) on to a SDS-PAGE gel or nitrocellulose membrane. As shown in FIG. 2, the plasma samples were subjected to sodium dodecyl sulfate-polyacrylamide gel electrophoresis (SDS-PAGE) and immunoblotting. After the proteins were transferred to the membrane, the membrane was blocked and later probed with rabbit-anti-Talin primary antibodies and goat-anti-rabbit-HRP. A HRP substrate was applied to the membranes, which was then applied to film and developed. The position of the Talin protein is indicated with an arrow (shown at right). It was found that Talin was only present in the HELA cells and the FSGS patient samples. Talin was absent from the control plasma samples. These results reinforce the finding, as shown in FIG. 1, that focal adhesion complex proteins are specific to FSGS patient plasma. Additionally, these results demonstrate that the focal adhesion complex proteins can be identified from FSGS plasma using similar antibody detection approaches (ELISA etc.), see FIG. 1B.

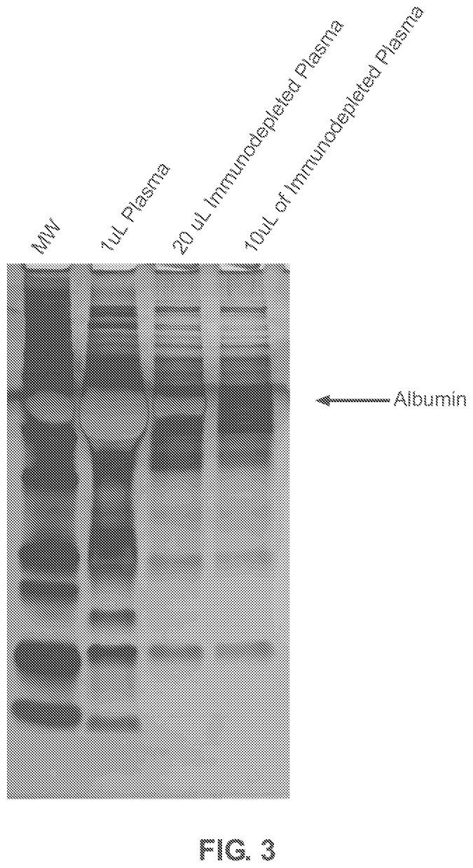

Referring now to FIG. 3, plasma proteins were subjected to Top-12 immunodepletion (a method designed to reduce the top-12 most abundant proteins from plasma or sera). The plasma proteins were either loaded directly onto an SDS-PAGE gel (1 uL Plasma (without immunodepletion)), 20 uL of immunodepleted plasma, or 10 uL of immunodepleted plasma. At 10 uL of immunodepleted plasma, it was found that the most abundant plasma proteins were reduced in concentration. Proteomics analysis was performed from 10 uL of immunodepleted plasma or sera.

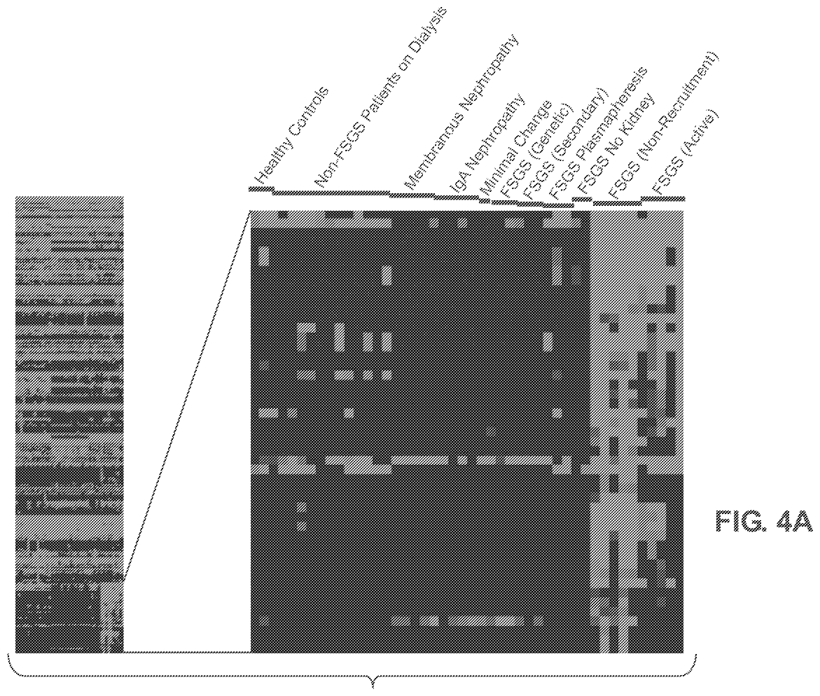

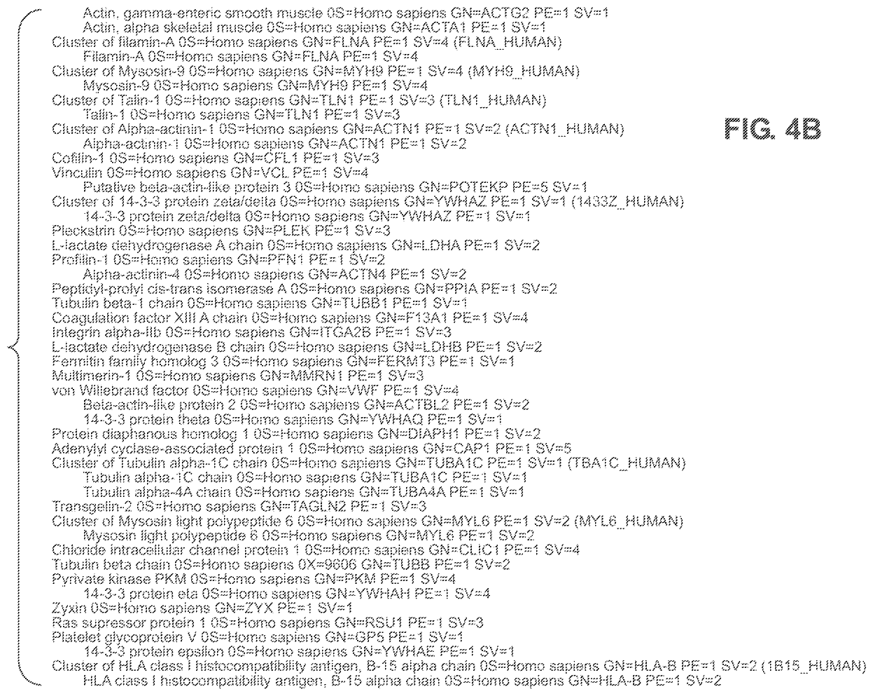

FIG. 4 uses hierarchical clustering to identify a group of focal adhesion complex proteins, specific to the patient population with a history of FSGS (n=10), that were not observed in control patient populations or patients with similar nephrotic diseases (n=36). These proteins are known components of the focal adhesion complex and include, but are not limited to: Filamin, Talin, Actinin, Cofilin, Vinculin, 14-3-3 protein, pleckstrin, Tubulin, Integrin, Fermitin, Multimerin, Diaphanous, Transgelin, Zyxin, and other proteins that are part of the focal adhesion complex. As shown in FIG. 4, a heatmap of immunodepleted proteins identified across plasma and sera from healthy controls, Non-FSGS patients on dialysis, patients with membranous nephropathy, patients with IgA nephropathy, a single patient with minimal change disease, patients with genetic FSSG, patients with secondary FSGS, patients with primary FSGS on plasmapheresis, patients with primary FSGS lacking a kidney (no kidney), patients with primary FSGS that received a transplant and are in a remission status (non-recurrent), and patients with active primary FSGS, was performed. The heatmap shows the presence of proteins (in red) or the absence of protein (in black) as normalized spectral values (defined by scaffold proteomics software). An amplified portion of the heatmap to the right of the figure shows proteins only observed in patients with subclinical (non-recurrent primary FSGS) and active primary FSGS.

Therefore, using the test kit provided, an ELISA or mass spectrometer may be used to assay specific protein levels in patients with nephrotic syndrome to monitor initial podocyte injury, disease progression, or therapeutic response achieved through treatment modalities. The kit may also be used 1) to diagnose FSGS and/or minimal change disease from other nephropathies, 2) as a prognostic indicator of treatment or transplant success and overall podocyte health, 3) as a secondary screen of patients that demonstrate abnormal proteinuria to identify FSGS/MCD, 4) to evaluate new or existing drug toxicity to podocytes by monitoring Focal Adhesion Complex Proteins during therapy, and 4) to test individuals at increased risk for developing podocytopathies, including people with APOL1 polymorphisms, medication-related toxicity, and other co-morbidities.

Specifically, with regard to distinguishing FSGS from other nephropathies, it has been found that if a patient presents with proteinuria and is unresponsive to immunosuppressive therapy, they can be tested for antibodies against PLA2R or Thrombospondin-1. Detection of antibodies against one or both of these proteins suggests the patient has membranous nephropathy.

Moreover, if the patient tests negative for the test described above, physicians can test for the presence of focal adhesion complex from bodily fluids. Testing positive for focal adhesion complex, but negative for antibodies against membranous nephropathy antigens (mentioned above), indicates that the patient has primary FSGS and should start immunosuppressive therapy and/or plasmapheresis.

Finally, if a patient presents with proteinuria and after renal biopsy, a diagnosis of FSGS may be made. Histologically primary, genetic, and secondary forms of FSGS are very difficult to differentiate. The presence of focal adhesion proteins in bodily fluid can confirm the diagnosis of primary FSGS and exclude the genetic and secondary forms. Therapy for these three forms of FSGS is completely different. Genetic and secondary forms do not require immunosuppressive therapy. Primary FSGS is currently treated with immunosuppressive therapy and plasma exchange.

### EXAMPLES

### Treatment Based on Diagnosis

Example 1—Diagnostic Utility: Patient presents with proteinuria, tests negative for all characterized forms of membranous nephropathy. Under these circumstances, physicians would test for the presence or absence of focal adhesion complex to further narrow the diagnosis of proteinuric nephropathy and/or confirm primary FSGS.

Example 2—Monitoring Response to Therapy: Patient presents with proteinuria and tests positive for focal adhesion complex proteins from a bodily fluid sample. The physician would immediately start immunosuppressive therapy including plasma exchange. Focal adhesion complex from bodily fluids could be used to monitor disease progression or severity.

Example 3—Monitoring Response to Therapy (specifically in the contest of using plasma exchange): A patient presents with proteinuria and tests positive for focal adhesion complex proteins from a bodily fluid sample. In addition to treating patients with immunosuppressive therapy, the physician might prescribe plasmapheresis. The physician might monitor levels of focal adhesion complex from bodily fluids as a prognostic indicator to determine if plasmapheresis is effective in slowing disease progression.

### Relationship to Antibodies

In addition to observing abundant focal adhesion complex in the plasma of FSGS patients, it can be surmised that components of the focal adhesion complex might be bound to antibodies. In order to better understand this relationship, immunoprecipitated antibodies from a patient with severe FSGS were tested using protein A/G magnetic beads (protein A/G binds to antibodies with high specificity).

As a negative control, from an identical amount of plasma, proteins that would bind to streptavidin beads (streptavidin has an affinity for biotin) were purified. A silver stain showed that the protein A/G purification successfully precipitated antibody heavy and light chain. It was found that the negative control purification (using streptavidin beads) was absent antibody heavy and light chain.

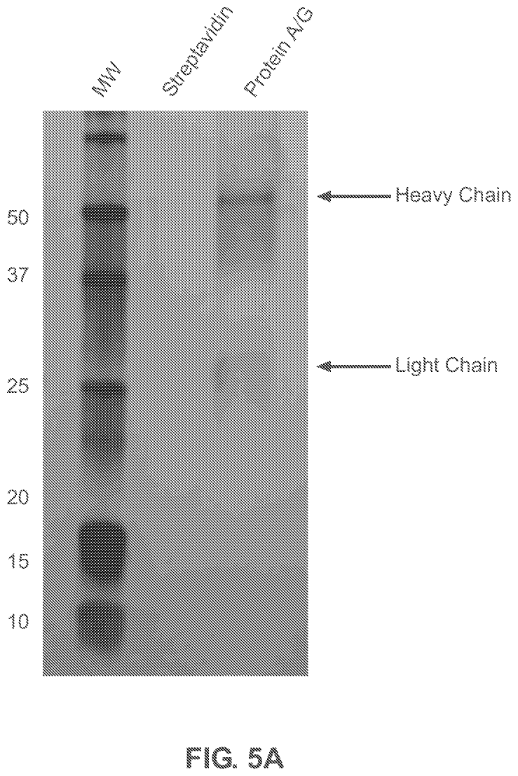

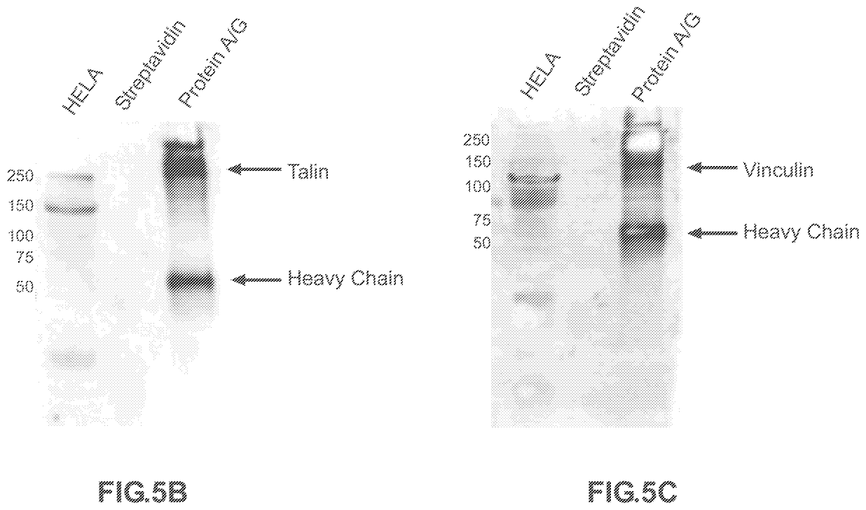

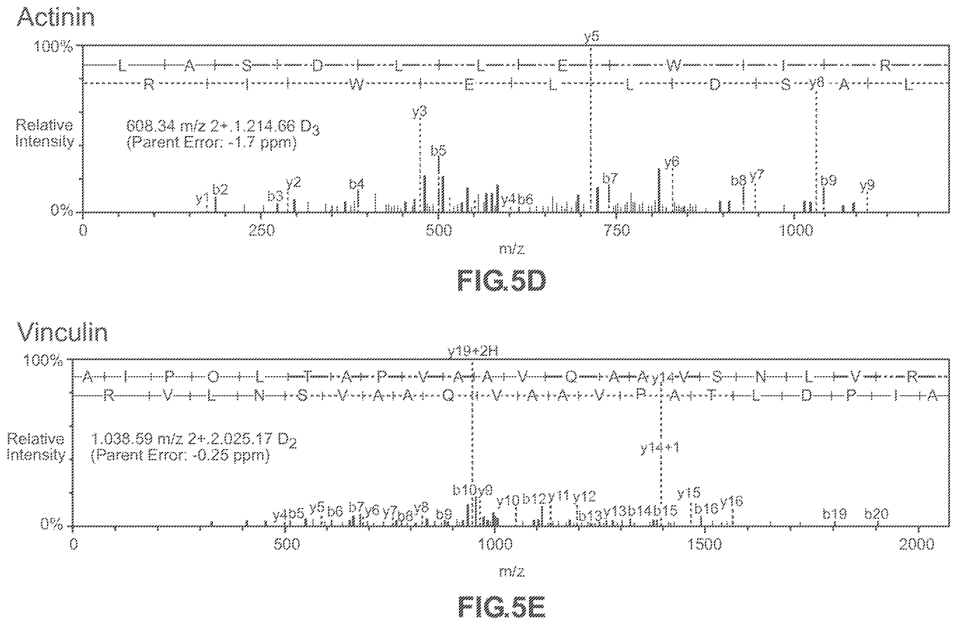

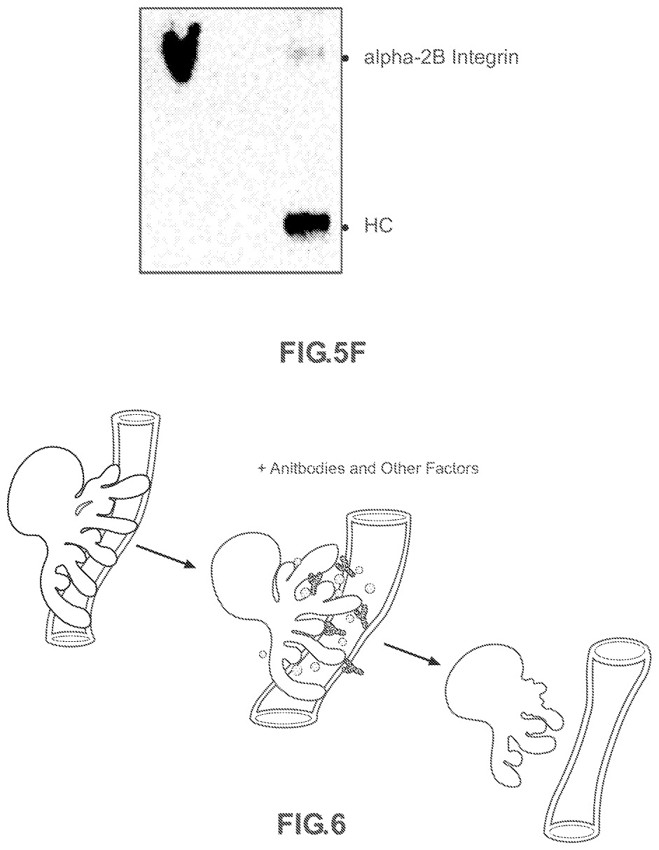

Following purification trypsinization of the proteins on the beads and mass spectrometry was performed to identify any additional proteins associated with the purified antibodies. Notably, the protein A/G purification, but not the streptavidin purification, contained peptides from the focal adhesion complex, as shown in FIG. 5. Additionally, immunoblotting showed that Talin and Vinculin were present in the protein A/G purification from FSGS plasma but were absent from the streptavidin purification. These results suggest that a population of FSGS patients have antibodies bound to their circulating focal adhesion complex.

Because antibodies may play a role in dissociating the podocyte foot process (largely composed on focal adhesion proteins), the antibodies targeting focal adhesion proteins might have a more powerful prognostic role than focal adhesion proteins alone. In one embodiment, physicians may treat patients that are positive for the focal adhesion complex with immunosuppressive therapy and then monitor antibody levels against the focal adhesion complex as a presumptive prognostic indicator.

In addition, that antibodies are bound to focal adhesion proteins from the podocytes suggests that the antibody-focal adhesion complex association is part of the FSGS disease mechanism. Therefore, the interaction between antibody and focal adhesion complex is a likely target for therapeutic intervention.

### Alternative Use for Diagnostic Test (Transplant, DOD)

Example 1: The US Army, Navy, Air Force, and Marine Corps perform comprehensive medical screens of recruits. In addition to the barrage of tests that are currently performed, each DOD agency would screen recruits for focal adhesion complex proteins among bodily fluids from candidates for enlistment or commission. A positive test would be used as a tool to flag future (or current) health concerns in military recruits.

Example 2: An organ transplant committee is establishing a prioritization list for kidney transplants. FSGS patients have ˜50% chance of recurrence after kidney transplant. Diagnosis of FSGS is difficult to differentiate from other diseases due to the absence of a clear molecular marker. Detection of focal adhesion complex in the bodily fluids of the transplant candidate would provide definitive and unambiguous evidence of FSGS. As a result, the transplant committee might consider evidence of focal adhesion complex in bodily fluids when determining kidney recipient priority given the likelihood of FSGS recurrence in renal allograft (when the focal adhesion complex is present). FSGS recurrence post renal transplant could be also monitored by measurement of focal adhesion proteins in bodily fluid to predict recurrence before proteinuria is clinically evident.

Example 3: Monitoring drug and treatment side-effect profiles that are toxic to podocytes. In humans medications often induce kidney stress that is poorly characterized. A subset of medications might induce podocyte effacement mimicking the damage induced by FSGS as a form of “podocytopathy”. As an example, puromycin induces FSGS in rodents. It is conceivable that other drugs will induce podocyte effacement, FSGS, and result in focal adhesion complex detection in the serum. As a result, monitoring focal adhesion complex in laboratory animals, patients receiving medication, or patients participating in a clinical trial for a new drug would be useful as a general marker of podocyte toxicity to indicate drug safety.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

1. A system for identifying damage or disease of a patient's kidney podocyte layer, wherein the system comprises a test kit configured to:
analyze the bodily fluid of the patient; and
identify the presence of focal adhesion complex proteins in the bodily fluid, wherein the focal adhesion complex proteins comprise Integrin, Talin, Filamin, Vinculin, and other components of the focal adhesion complex.

2. A method for identifying damage or disease of a patient's kidney podocyte layer, wherein the method comprises:
providing a test kit configured to analyze the bodily fluid of the patient;
using the test kit to analyze the bodily fluid of the patient; and
identifying the presence of focal adhesion complex proteins in the patient's plasma, wherein the focal adhesion complex proteins comprise Integrin, Talin, Filamin, Vinculin, or other focal adhesion complex proteins.

3. A system for identifying or predicting damage or disease of a patient's kidney podocyte layer, wherein the system comprises a test kit configured to:
analyze the bodily fluid of the patient; and
identify the presence of anti-bodies associated with focal adhesion complex proteins in the bodily fluid, wherein the focal adhesion complex proteins comprise Integrin, Talin, Filamin, Vinculin, and other components of the focal adhesion complex.

4. A method for identifying or predicting damage or disease of a patient's kidney podocyte layer, wherein the method comprises:
providing a test kit configured to analyze the bodily fluid of the patient;
using the test kit to analyze the bodily fluid of the patient; and
identifying the presence of antibodies associated with focal adhesion complex proteins in the patient's bodily fluid, wherein the focal adhesion complex proteins comprise Integrin, Talin, Filamin, Vinculin, or other focal adhesion complex proteins.
